(19) United States
(12) Patent Application Publication
(10) Pub. No.: US 2019/0091026 A1
(43) Pub. Date: Mar. 28, 2019

(54) SPINAL CORD PROTECTING APPARATUS

(71) Applicant: MEDYSSEY CO., LTD., Jecheon-si (KR)

(72) Inventors: Jong-Wuk JANG, Seoul (KR); Su Heon WOO, Uijeongbu-si (KR); K. Daniel RIEW, New York, NY (US)

(73) Assignee: MEDYSSEY CO., LTD., Jecheon-si (KR)

(21) Appl. No.: 16/141,633

(22) Filed: Sep. 25, 2018

(30) Foreign Application Priority Data
Sep. 26, 2017 (KR) 10-2017-0123888

(51) Int. Cl. A61F 2/30; A61L 27/56; A61F 2/44

(52) U.S. Cl. CPC A61F 2/30721; A61L 27/56; A61B 17/7071; A61F 2/30767; A61F 2220/0025; A61F 2/44

(57) ABSTRACT

Disclosed is a spinal cord protecting apparatus. The spinal cord protecting apparatus includes a body and a fixture. The body is formed in a porous plate shape to cover an exposed spinal cord. The fixture is configured to fix the body. Accordingly, the spinal cord exposed to the outside is safely protected, so that an additional disease is prevented.

### CROSS-REFERENCE TO RELATED APPLICATIONS

This application claims priority to and the benefit of Korean Patent Application No. 10-2017-0123888 filed in the Korean Intellectual Property Office on Sep. 26, 2017, the entire contents of which are incorporated herein by reference.

### BACKGROUND OF THE INVENTION

### 1. Field of the Invention

The present invention relates to a spinal cord protecting apparatus, and more particularly, to a spinal cord protecting apparatus for covering a spinal cord exposed when the surgery is performed to a vertebra.

### 2. Description of the Related Art

Referring to FIG. 1, a spinal cord 4 extends between vertebrae 2 of a person 1. The spinal cord has a length of 41 cm to 45 cm, and includes motor nerves, sensory nerves, and autonomic nerves. When a space S-1 of a passage for the spinal cord 4 becomes narrow due to discopathy (a disc disease), degenerative spinal stenosis or the like, the spinal cord 4 is physically compressed. When the spinal cord 4 is compressed, a symptom of myelopathy or radiculopathy appears, and thus a patient feels pain.

Laminectomy for removing a lamina 3 is performed to widen the space S-1 of the narrowed spinal cord 4. FIG. 1 shows a process of removing and taking out the lamina 3 from the vertebra.

Since the laminectomy entirely removes the lamina 3, the vertebra 2 becomes unstable and additional degenerative diseases are induced. To redeem the problems caused by the laminectomy, laminoplasty has been proposed to spread the lamina in order to widen the space through which the spinal cord 4 extends. Referring to FIG. 2, the laminoplasty forms one end 5 by cutting a predetermined portion of the lamina 3. Further, opposite one end is dug to form a hinge 6 and the one end 5 is lifted to widen the space.

However, the spinal cord 4 is exposed to the outside when performing the laminectomy and the laminoplasty. Although the spinal cord 4 exposed to the outside is covered with muscles, the spinal cord 4 protected by a hard bone is exposed, so the protection only by thin muscles causes anxiety to the patient. In addition, external compression may be applied to the spinal cord 4 or another additional disease may be caused. In addition, a doctor performing surgery on another part is required to be careful not to touch the exposed spinal cord 4.

### SUMMARY OF THE INVENTION

To solve the above problems, the present invention provides a spinal cord protecting apparatus for protecting an exposed spinal cord.

According to an embodiment of the present invention to solve the above problems, there is provided a plate-shape spinal cord protecting apparatus formed therein with a plurality of holes so as to cover the exposed spinal cord. Accordingly, the spinal cord exposed to the outside is safely protected so that the patient can be relieved and an additional disease is prevented.

The ratio of the maximum longitudinal length to the maximum lateral length may be 1:1.5 to 1:3.

A thickness may be 3 mm to 8 mm.

At least some of the holes may fix the spinal cord protecting apparatus.

Opened slots extending from both ends of the spinal cord protecting apparatus and engaged with screws may be further included.

The spinal cord protecting apparatus may be bent in correspondence with the protruding degree of the spinal cord.

A plurality of grooves may be provided on a surface thereof.

At least one opening may be formed.

A body is coupled to an implant through the opening, in which the implant is formed in a semicircular shape and a protrusion may extend outward from a center of the implant.

According to an embodiment of the present invention, the spinal cord exposed to the outside is safely protected so that the patient can be relieved and the additional disease can be prevented due to the exposure of the spinal cord.

In addition, according to an embodiment of the present invention, the spinal cord protecting apparatus is coupled to the implant instead of the lamina removed by the laminectomy, so that the instability of the vertebrae can be removed and an additional degenerative disease can be prevented.

Further, according to an embodiment of the present invention, a passage in which the spinal cord extends is sufficiently secured, so that the problem that the spinal cord is compressed to cause pain can be resolved.

### BRIEF DESCRIPTION OF THE DRAWINGS

FIG. 1 shows an example of a process of performing laminectomy of removing a lamina from a vertebra to secure a space of a passage for a spine cord.

FIG. 2 shows an example of a process of performing laminoplasty to secure a space of a passage for a spine cord without removing a lamina from a vertebra.

FIG. 3 shows a spinal cord protecting apparatus according to an embodiment of the present invention.

FIG. 4 shows spinal cord protecting apparatuses having different curvatures according to another embodiment of the present invention.

FIG. 5 shows a usage example of a spinal cord protecting apparatus according to an embodiment of the present invention.

FIG. 6 shows a usage example of a spinal cord protecting apparatus according to another embodiment of the present invention.

FIG. 7 shows a spinal cord protecting apparatus coupled to an implant according to an embodiment of the present invention.

FIG. 8 shows a spinal cord protecting apparatus coupled to an implant according to an embodiment of the present invention.

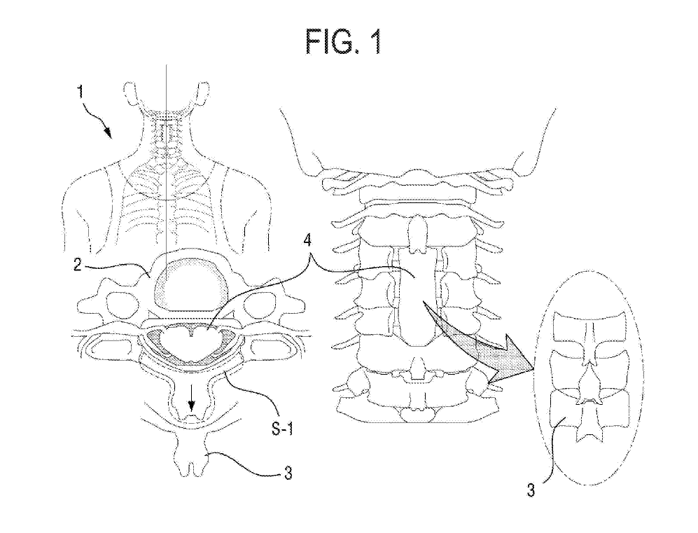

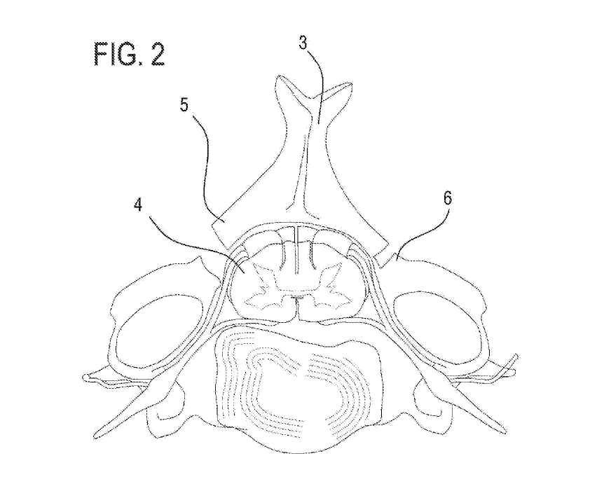

### DETAILED DESCRIPTION OF THE INVENTION

Hereinafter, a preferred embodiment of the present invention will be described in detail with reference to the accompanying drawings.

Advantages and features, and methods of achieving the advantages and features will be apparent with reference to the embodiments described in detail below with reference to the accompanying drawings. However, the present invention is not limited to the embodiments described as below and it may be implemented in various different forms. The embodiments are provided to complete the disclosure of the present invention and clearly teach the scope of the invention to those skilled in the art of the present invention, therefore, the present invention will be defined only by the scope of claims. The same reference numeral indicates the same element throughout the specification.

Unless otherwise defined, all terms (including technical and scientific terms) used herein may be used in a sense commonly understood by those skilled in the art of the present invention. In addition, terms defined in generally used dictionaries are not ideally or excessively interpreted unless explicitly defined otherwise. The terms used herein is for the purpose of illustrating embodiments and the present invention is not limited thereto. Herein, a singular form includes a plural form unless the context is particularly stated otherwise.

The terms used herein is for the purpose of illustrating embodiments and the present invention is not limited thereto. In addition, the term “comprise” and/or “include” used herein specifies the presence of mentioned shapes, numbers, steps, members, elements and/or groups thereof, and it is not intended to exclude the presence or addition of other unmentioned shapes, numbers, operations, members, elements and/or groups thereof.

In addition, a structure or shape disposed adjacent to another shape may have a portion overlapping the adjacent shape or disposed on a lower part of the adjacent shape.

Relative terms herein such as below, above, upper, lower, horizontal, and vertical may be used to describe a relationship between a constituent member, layer or region and another constituent member, layer or region, as shown in the drawings. The above terms cover not only the directions indicated in the drawings but also the other directions of the apparatus.

Hereinafter, embodiments of the present invention will be described with reference to cross sectional views schematically showing ideal embodiments (and intermediate structures) of the present invention. In these drawings, for example, sizes and shapes of members may be exaggerated for convenience and clarity of description, and deformations of the depicted shapes upon actual implementation may be anticipated. Therefore, embodiments of the present invention are not limited to any particular shape of regions shown herein.

The spinal cord protecting apparatus according to an embodiment of the present invention is coupled while covering a spinal cord exposed after laminoplasty or laminectomy.

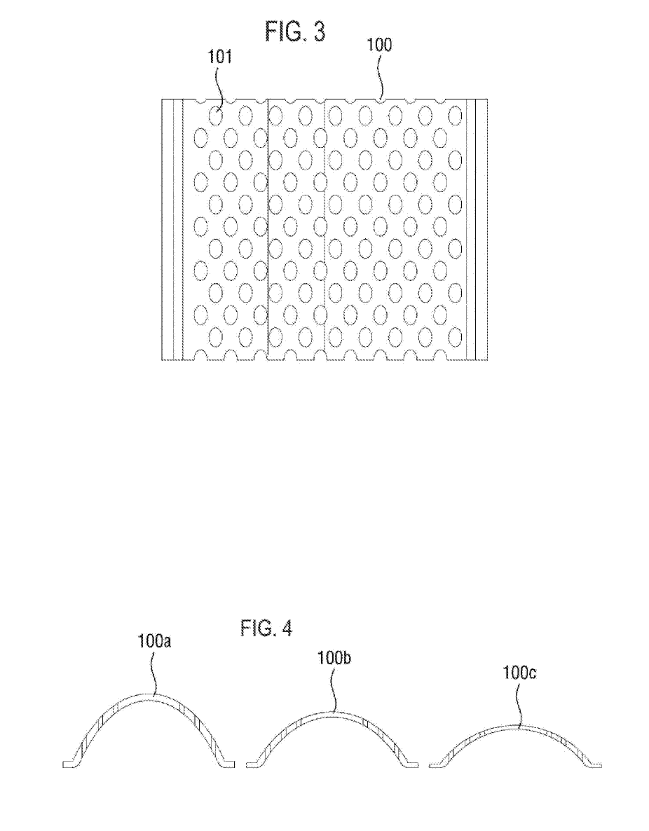

FIG. 3 shows a spinal cord protecting apparatus according to an embodiment of the present invention.

The spinal cord protecting apparatus 100 is configured to have a plate shape formed therein with a plurality of holes 101. The holes 101 formed in a body of the spinal cord protecting apparatus 100 may be used to fix the spinal cord protecting apparatus 100.

In addition, the holes 101 prevent bone/muscle positioned above/below the spinal cord protecting apparatus 100 from being completely isolated, so that the user feels less foreign body sensation. In addition, human body tissues coming into contact with each other between the holes 101 are combined or agglutinated with each other, so that the spinal cord protecting apparatus 100 is completely fixed to the human body. Further, the spinal cord protecting apparatus 100 is configured to have a porous structure including the holes 101 so as to be more easily cut into a required shape.

The body of the spinal cord protecting apparatus 100 has a thickness of 3 mm to 8 mm, and preferably about 3.5 mm to about 5 mm.

A thickness for the body of the spinal cord protecting apparatus 100 is properly selected to be easily bent while maintaining a shape in a body of the patient when force is applied to the spinal cord protecting apparatus 100. In addition, the thickness for the spinal cord protecting apparatus 100 may be properly selected to be easily cut by scissors or the like as required.

The spinal cord protecting apparatus 100 has different sizes and shapes depending on a fixed position, an appearance of fixed body tissue, or the like. The user may measure an area of the spinal cord to be covered, measure a size of the spinal cord protecting apparatus 100 based on the measured area, and produce the spinal cord protecting 100. The ratio of the longitudinal length to the lateral length of the spinal cord protecting apparatus 100 may be 1:1.5 to 1:3 for smooth protection.

The spinal cord protecting apparatus 100 may be formed of various materials such as titanium (Titanium-Tit6Al4v-ELI, Ti6Al4v or Grade 2), PEEK, and titanium alloy. In addition, a plurality of grooves and/or a plurality of protrusions may be formed on a surface thereof, so that the surface is rough as a whole. Since the surface of the spinal cord protecting apparatus 100 is roughly formed as a concavo-convex surface, a surface area may become wider, so that the spinal cord protecting apparatus 100 can be effectively attached to a fixed bone.

The spinal cord protecting apparatus 100 may be provided at an outer side thereof with a porous cover (a mesh cover) to surround the spinal cord protecting apparatus 100. The spinal cord protecting apparatus 100 not only protects the spinal cord, but also provides convenience during bone grafting.

FIG. 4 shows spinal cord protecting apparatuses having different curvatures according to another embodiment of the present invention.

The spinal cord protecting apparatuses 100a, 100b, and 100c may have different curvatures according to a size and shape of a vertebra positioned in a spinal cord to be covered. For example, more curved spinal cord protecting apparatus 100a, 100b, and 100c may be used when the spinal cord further protrudes from the vertebra outward.

The user may bend and use the plate-shape spinal cord protecting apparatus 100a, 100b, and 100c to have a suitable curvature.

In another embodiment of the present invention, the spinal cord protecting apparatus 100a, 100b and 100c may be produced to have a different curvature during production process.

The spinal cord protecting apparatus 100a, 100b and 100c may be bent in various shapes and directions unlike the drawing. For example, the spinal cord protecting apparatus 100a, 100b and 100c may have a plurality of convex or concave portions. The drawing is shown merely for illustrative convenience and it is not intended to limit the size and the direction and degree of bending of the spinal cord protecting apparatus 100a, 100b and 100c.

Hereinafter, an actual usage example of the spinal cord protecting apparatus 100 will be described with reference to drawings. To avoid redundant description, it is described that the spinal cord protecting apparatus 100 is coupled after the lamina 3 is completely removed by the laminectomy, however, the spinal cord protecting apparatus 100 according to an embodiment of the present invention also may be used to cover a partially exposed spinal cord 4 after the lamina 3 is lifted by the laminoplasty.

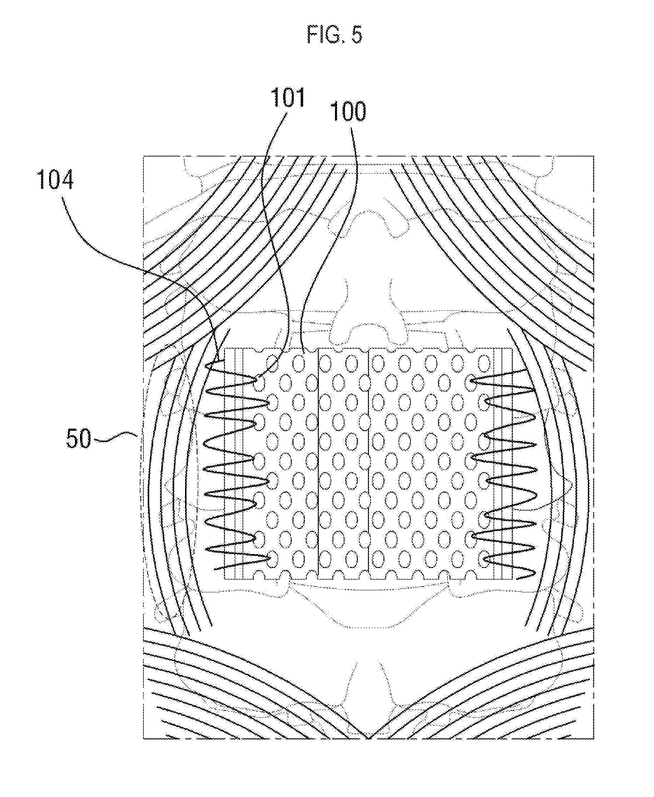

FIG. 5 shows a usage example of a spinal cord protecting apparatus according to an embodiment of the present invention.

Referring to FIG. 5, the spinal cord protecting apparatus 100 is mounted at a site in which the lamina at a rear side of the vertebra is removed. The lamina is removed from the vertebra and the spinal cord protecting apparatus 100 covers the spinal cord to cover the exposed spinal cord. The spinal cord protecting apparatus 100 is configured to cover and protect the exposed spinal cord. A suture 104 passes through at least one of the holes 101 in the body of the spinal cord protecting apparatus 100, such that the spinal cord protecting apparatus 100 is stitched and fixed to muscles 50. The hole 101 through which the suture 104 is passed serves as a fixture of the spinal cord protecting apparatus 100.

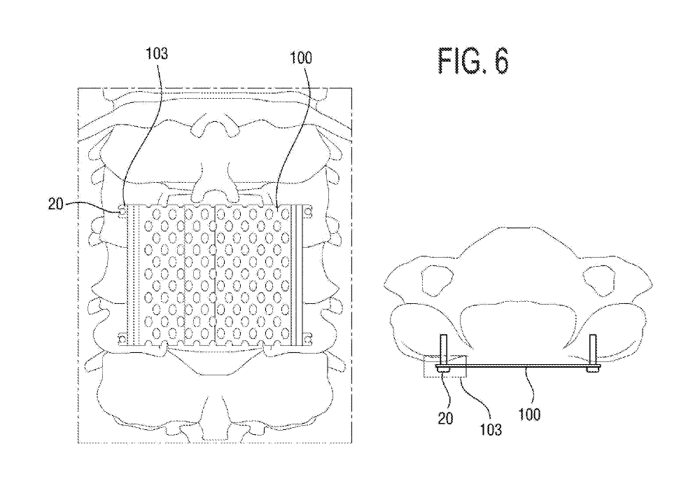

FIG. 6 shows a usage example of a spinal cord protecting apparatus according to another embodiment of the present invention.

The spinal cord protecting apparatus 100 may be fixed to both ends of the rear side of the vertebra by using screws 20. To this end, the spinal cord protecting apparatus 100 is configured to extend from both ends of the body and includes opened slots 103 coupled to the screws 20.

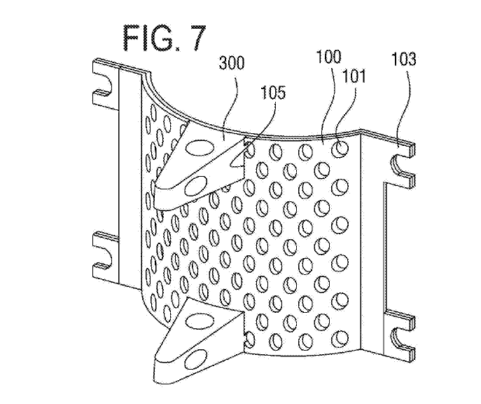

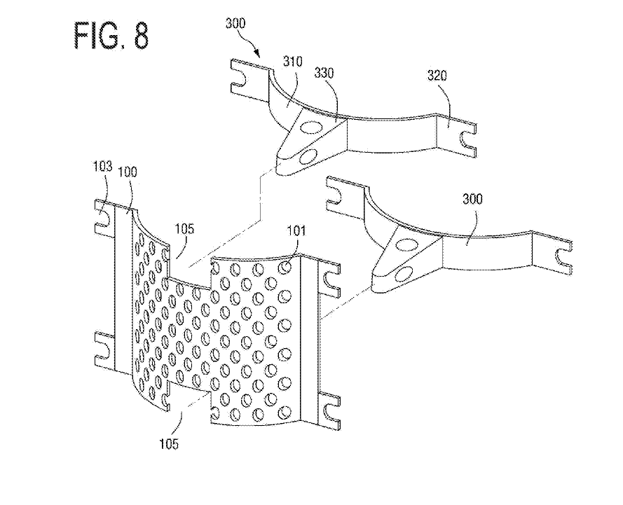

FIGS. 7 and 8 show a spinal cord protecting apparatus coupled to an implant according to an embodiment of the present invention.

According to an embodiment of the present invention, the spinal cord protecting apparatus 100 is used in combination with the implant 300 for covering the lamina removal site where the laminae is removed by performing the laminectomy. According to another embodiment of the present invention, even when the lamina is lifted after the laminoplasty is performed, the spinal cord protecting apparatus 100 may be used in combination with the implant 300 for fixing the lifted lamina.

The implant 300 may be fixed by the screws 20. The implant 300 has a semicircular body having a predetermined width and thickness and includes a fixture provided with an opened slot to be coupled to the screw 20. Further, the implant 300 may further include a protrusion extending outward from a center thereof. The implant 300 is fixed to the vertebra and serves to support the vertebra to prevent degeneration or fracture.

At least one opening 105 coupled to the implant 300 may be formed in the body of the spinal cord protecting apparatus 100. The opening 105 may be formed by cutting the body by the user. A shape of the opening 105 is not limited to the illustrated contents and may be configured in various shapes. A cover member may be provided on an inner side of the opening 105 to protect the human body from the cut surface of the opening 105.

The opening 105 is not limited to being formed at the outer side of the spinal cord protecting apparatus 100, as shown in the drawing. In another embodiment of the present invention, the opening 105 may be formed to avoid contact with another body tissue such as bone, blood vessel and nerve. As an example, the opening 105 may be formed at inner side of the spinal cord protecting apparatus 100. According to the embodiment, human body tissues positioned over and below of the spinal cord protecting apparatus 100 may pass through the opening 105 and be fused to each other.

According to the above embodiment, the spinal cord protecting apparatus 100 is used together with the implant 300, so that a space through which the spinal cord passes is sufficiently secured. Further, degenerative diseases that may occur after partial or complete removal of the lamina are prevented.

HA coating or plasma coating may be performed on a portion in which a bone comes into contact with the implant 300 or the spinal cord protecting apparatus 100.

It shall be apparent to those skilled in the art that the present invention is not limited to the described embodiments and accompanying drawings, and various replacements, deformations and modifications are available without departing from the spirit or scope of the invention as defined in the appended claims.

What is claimed is:

1. A spinal cord protecting apparatus having a plate shape and formed therein with a plurality of holes so as to cover an exposed spinal cord.

2. The spinal cord protecting apparatus of claim 1, wherein the spinal cord protecting apparatus comprises a body that covers the spinal cord exposed through a lamina incision site at a rear side of a vertebra.

3. The spinal cord protecting apparatus of claim 1, wherein a ratio of a maximum longitudinal length to a maximum lateral length is 1:1.5 to 1:3.

4. The spinal cord protecting apparatus of claim 1, wherein spinal cord protecting apparatus has a thickness of 3 mm to 8 mm.

5. The spinal cord protecting apparatus of claim 1, wherein at least some of the holes are configured to fix the spinal cord protecting apparatus.

6. The spinal cord protecting apparatus of claim 1, wherein opened slots extend from both ends of the spinal cord protecting apparatus so as to be coupled to screws.

7. The spinal cord protecting apparatus of claim 1, wherein the spinal cord protecting apparatus is bent in correspondence with a protruding degree of the spinal cord.

8. The spinal cord protecting apparatus of claim 1, wherein a plurality of grooves are formed on a surface of the spinal cord protecting apparatus.

9. The spinal cord protecting apparatus of claim 1, wherein at least one opening is formed in the spinal cord protecting apparatus.

10. The spinal cord protecting apparatus of claim 9, wherein the spinal cord protecting apparatus comprises a body coupled to an implant through the opening, in which the implant is formed in a semicircular shape and a protrusion extends outward from a center of the implant.
